(19) United States
(12) Patent Application Publication
(10) Pub. No.: US 2026/0092912 A1
(43) Pub. Date: Apr. 2, 2026

(54) DOWNHOLE MEASUREMENT SYSTEM

(71) Applicant: Schlumberger Technology Corporation, Sugar Land, TX (US)

(72) Inventor: Richard Bloemenkamp, Clamart (FR)

(21) Appl. No.: 18/900,952

(22) Filed: Sep. 30, 2024

(51) Int. Cl. G01V 3/26; E21B 47/12

(52) U.S. Cl. CPC G01N 33/2823; E21B 47/113; G01N 27/06; G01N 27/221

(57) ABSTRACT

A system includes an arm portion of a tool string to be lowered into a wellbore and a measurement system. The measurement system includes a first electrode coupled to an inner surface of the arm portion, a first bracket coupled to the inner surface, and a second electrode coupled to a portion of the first bracket. The second electrode and the portion of the first bracket are offset from and normal to a conductive surface of the first electrode. The second electrode and the portion of the first bracket are at least partially within an outer perimeter of the conductive surface.

### BACKGROUND

This disclosure relates to systems and methods for a downhole measurement system used in wellbores.

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as an admission of any kind.

During the process of extracting hydrocarbons from drilled wells, measurements of one or more characteristics of a well fluid may be obtained using a measurement system. One method of obtaining these well fluid measurements involves measuring electrical properties of the well fluid using an electrode. Overtime, debris from the well fluid may eventually cause fouling and/or clogging of portions of the electrode. Accordingly, a downhole measurement system that measures electrical properties of well fluid while mitigating clogging and/or fouling is desired.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In an embodiment, a system includes an arm portion of a tool string to be lowered into a wellbore and a measurement system. The measurement system includes a first electrode coupled to an inner surface of the arm portion, a bracket coupled to the inner surface, and a second electrode coupled to a portion of the bracket. The second electrode and the portion of the bracket are offset from a conductive surface of the first electrode. The second electrode and the portion of the bracket are at least partially within an outer perimeter of the conductive surface.

In another embodiment, a downhole measurement system includes a first electrode that couples to an inner surface of an arm portion of a tool string, a first bracket that couples to the inner surface, and a second electrode that couples to a portion of the first bracket. The second electrode and the portion of the first bracket, when coupled to the inner surface, are offset from a conductive surface of the first electrode. The second electrode and the portion, when coupled to the inner surface, are disposed within an outer perimeter of the conductive surface.

In another embodiment, a system includes a downhole measurement assembly kit. The downhole measurement assembly kit includes at least one of a first electrode that couples to an inner surface of an arm portion of a tool string, a bracket that couples to the inner surface, and a second electrode that couples to a portion of the bracket. The second electrode and the portion of the bracket, when coupled to the inner surface, are offset from a conductive surface of the first electrode. The second electrode and the portion of the bracket, when coupled to the inner surface, are within an outer perimeter of the conductive surface.

Various refinements of the features noted above may be undertaken in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended to familiarize the reader with certain aspects and contexts of embodiments of the present disclosure without limitation to the claimed subject matter.

### BRIEF DESCRIPTION OF THE DRAWINGS

Various aspects of this disclosure may be better understood upon reading the following detailed description and upon reference to the drawings in which:

FIG. 1 is a schematic diagram of a well system having a downhole measurement system, in accordance with an embodiment of the present disclosure;

FIG. 2 is a perspective view of a tool string that includes the downhole measurement system of FIG. 1 coupled to a downhole caliper system, in accordance with an embodiment of the present disclosure;

FIG. 3 is a side schematic view of the downhole measurement system of FIG. 1 having multiple measurement assemblies, in accordance with an embodiment of the present disclosure;

FIG. 4 is a perspective close-up view of a downhole measurement assembly of the downhole measurement system of FIG. 3 within an area identified by line 4-4, in accordance with an embodiment of the present disclosure;

FIG. 5 is a cross-sectional view of the downhole measurement assembly of FIG. 3 within an area identified by line 5-5 when the downhole caliper system is in an open configuration, in accordance with an embodiment of the present disclosure;

FIG. 6 is a cross-sectional view of the downhole measurement assembly of FIG. 3 with the downhole caliper system in a closed configuration, in accordance with an embodiment of the present disclosure;

FIG. 7 is a cross-sectional view of the downhole measurement assembly of FIG. 3 with the downhole caliper system in a closed configuration and a first electrode of the downhole measurement assembly formed into the tool string, in accordance with an embodiment of the present disclosure;

FIG. 8 is a series of cross-sectional views of a second electrode of the downhole measurement assembly, in accordance with an embodiment of the present disclosure; and

FIG. 9 is a perspective close-up view of the downhole measurement assembly of the downhole measurement system of FIG. 3 within an area identified by line 9-9, in accordance with an embodiment of the present disclosure.

### DETAILED DESCRIPTION

Certain embodiments commensurate in scope with the present disclosure are summarized below. These embodiments are not intended to limit the scope of the disclosure, but rather these embodiments are intended only to provide a brief summary of certain disclosed embodiments. Indeed, the present disclosure may encompass a variety of forms that may be similar to or different from the embodiments set forth below.

As used herein, the term “coupled” or “coupled to” may indicate establishing either a direct or indirect connection (e.g., where the connection may not include or include intermediate or intervening components between those coupled), and is not limited to either unless expressly referenced as such. The term “set” may refer to one or more items. Wherever possible, like or identical reference numerals are used in the figures to identify common or the same elements. The figures are not necessarily to scale and certain features and certain views of the figures may be shown exaggerated in scale for purposes of clarification.

Furthermore, when introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment,” “an embodiment,” or “some embodiments” of the present disclosure are not intended to be interpreted as excluding the existence of additional embodiments that also incorporate the recited features. Furthermore, the phrase A “based on” B is intended to mean that A is at least partially based on B. Moreover, unless expressly stated otherwise, the term “or” is intended to be inclusive (e.g., logical OR) and not exclusive (e.g., logical XOR). In other words, the phrase A “or” B is intended to mean A, B, or both A and B.

Embodiments of the present disclosure relate to systems for a downhole measurement system lowered into wellbores for measuring electrical properties of a well fluid. Based on these measured electrical properties, a composition of the well fluid may be estimated. The downhole measurement system includes a first electrode coupled to an inner surface of an arm of a tool string, a bracket coupled to the inner surface of the tool string, and a second electrode coupled to the bracket such that at least a portion of the second electrode is offset from the first electrode and falls within an outer perimeter of a conductive portion of the first electrode. The second electrode includes electrical shielding disposed in an outer radial portion of the second electrode to mitigate an effect of the tool string spine on measurements obtained from the downhole measurement system.

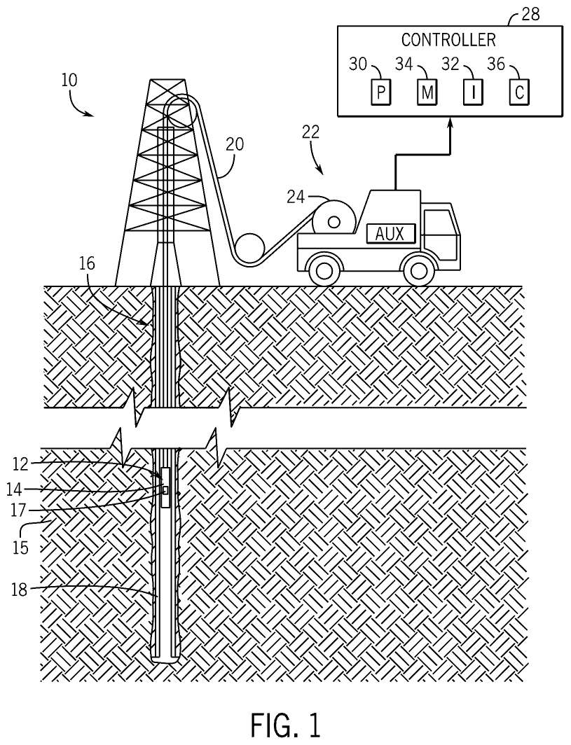

With the foregoing in mind, FIG. 1 is a schematic diagram of a well system 10 having a downhole measurement system 12. The well system 10 may be used to convey a tool string 14 that includes the downhole measurement system 12 through a geological formation 15 via a wellbore 16. In certain embodiments, a casing 18 may be disposed within the wellbore 16, such that the tool string 14 may traverse the wellbore 16 within the casing 18. As described in further detail herein, the downhole measurement system 12 may be used to make one or more measurements within the wellbore 16. In the illustrated embodiment, the tool string 14 includes a downhole caliper system 17 that extends and/or retracts from the tool string 14. In certain embodiments, the downhole measurement system 12 may be coupled to the downhole caliper system 17. The tool string 14 may be conveyed on a conveyance cable 20 via a conveyance cable spooling system 22. In certain embodiments, the tool string 14 may be conveyed via a drill-pipe and/or coil tubing. Although the conveyance cable spooling system 22 is schematically shown in FIG. 1 as a mobile cable spooling system carried by a truck, the conveyance cable spooling system 22 may instead be substantially fixed (e.g., a long-term installation that is substantially permanent or modular). Any conveyance cable 20 suitable for conveying the tool string 14 may be used. The conveyance cable 20 may be spooled and unspooled on a spool 24 and an auxiliary power source 26 may provide energy to the conveyance cable spooling system 22, the tool string 14, and/or the downhole caliper system 17.

In certain embodiments, the downhole measurement system 12 may include a controller 28 via any suitable telemetry (e.g., via electrical or optical signals pulsed through the conveyance cable 20, or through the geological formation 15 or via mud pulse telemetry). The controller 28 may be any electronic data processing system that can be used to carry out the functionality described herein. For example, the controller 28 may include one or more processors 30, which may execute instructions 32 stored in memory 34 via circuitry 36. As such, the memory 34 of the controller 28 may be any suitable article of manufacture that can store the instructions 32. The memory 34 may be ROM memory, random-access memory (RAM), flash memory, an optical storage medium, or a hard disk drive, to name a few examples. In certain embodiments, the downhole measurement system 12 may include a power source 38 that may power the controller 28, the downhole caliper system 17, and/or the downhole measurement system 12. In certain embodiments, the controller 28 may be disposed within the tool string 14, such that data stored on the memory 34 may be transmitted to the surface via a cable and/or retrieved when the tool string 14 is brought to the surface.

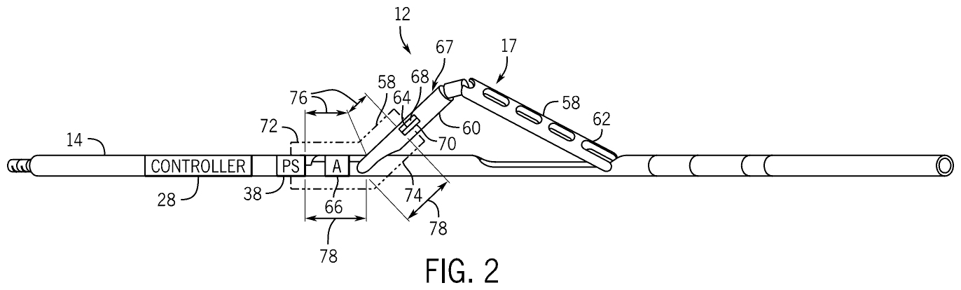

FIG. 2 is a perspective view of the tool string 14 including the downhole measurement system 12 and the downhole caliper system 17. In the illustrated embodiment, the downhole caliper system 17 includes arms 58 (e.g., first arm 60, second arm 62). As shown, the first arm 60 (e.g., first arm portion) and the second arm 62 (e.g., second arm portion) are rotably coupled to the tool string 14. In the illustrated embodiment, the downhole measurement system 12 includes a measurement assembly 64 (e.g., downhole measurement assembly) coupled to the first arm 60 of the downhole caliper system 17. Additionally or alternatively, the measurement assembly 64 may be coupled to the second arm 62 and/or the tool string 14. As shown, the controller 28 and the power source 38 of the downhole measurement system 12 are disposed in the tool string 14. In certain embodiments, the downhole measurement system 12 includes an amplifier 66. In the illustrated embodiment, the amplifier 66 is disposed in the tool string 14.

In the illustrated embodiment, the measurement assembly 64 includes a pair of electrodes 67 including a first electrode 68 and a second electrode 70. In certain embodiments, the first electrode 68 is a receiver electrode and the second electrode 70 is a transmitter electrode. Additionally or alternatively, the first electrode 68 may be a transmitter electrode and the second electrode 70 may be a receiver electrode. In certain embodiments, a current flows from the first electrode 68 to the second electrode 70. Additionally or alternatively, the current may flow from the second electrode 70 to the first electrode 68. The current may be a direct current (DC) or an alternating current (AC) signal. As shown, the first electrode 68 and the second electrode 70 are coupled to the first arm 60. Additionally or alternatively, the pair of electrodes 67 may be coupled to the second arm 62 and/or the tool string 14.

In the illustrated embodiment, the first electrode 68 is electrically coupled to the power source 38 (e.g., receiver circuitry and/or transmitter circuitry) via a first wire 72 (e.g., coaxial cable) and the second electrode 70 is electrically coupled to the power source 38 via a second wire 74 (e.g., coaxial cable). In certain embodiments, a first length 76 of the first wire 72, a second length 78 of the second wire 74, or a combination thereof is greater than 50 centimeters (cm). For example, the first length 76, the second length 78, or both may be greater than 60 cm, 70 cm, 80 cm, 90 cm, 100 cm, 200 cm, or 500 cm. In certain embodiments, the first wire 72, the second wire 74, or a combination thereof is shielded and electrically coupled to the amplifier 66. An operating frequency of the first electrode 68, the second electrode 70, or both may be between 20 kilohertz (kHz) and 10 megahertz (MHz). In certain embodiments, the operating frequency of the first electrode 68, the second electrode 70, or both may be between 500 kHz and 2 MHz.

The controller 28 may receive a signal from the first electrode 68 (e.g., receiver electrode) indicative of a dielectric permittivity, capacitance, and/or conductivity of a fluid disposed in the wellbore. The controller 28 may determine an estimated capacitance of the fluid based on the received signal. In certain embodiments, the controller 28 may measure an amplitude, a phase shift, or both of a current received from the first electrode 68 with respect to a current sent to the second electrode 70 (e.g., transmitter electrode). It may be appreciated that by estimating the capacitance of the fluid between the pair of electrodes 67, the controller 28 may distinguish between oil-continuous and water-continuous mixtures.

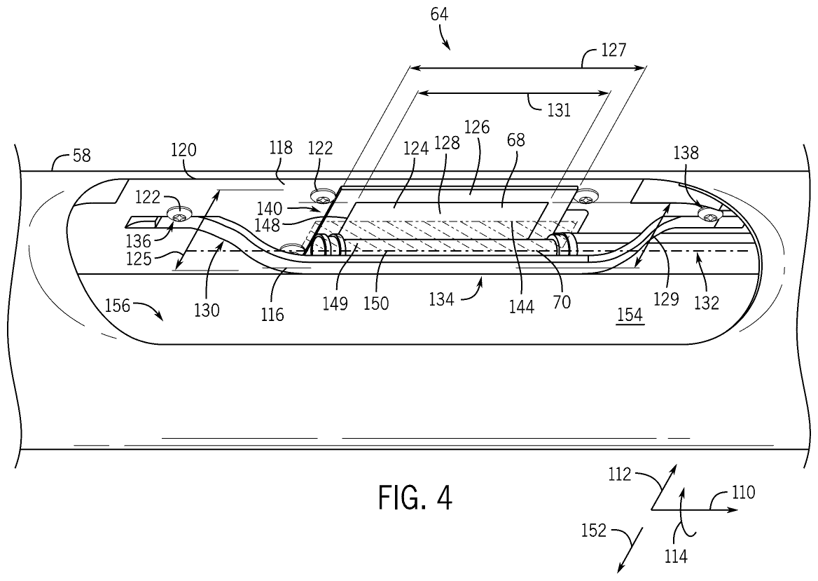

In certain embodiments, the measurement assembly 64 may be a downhole measurement assembly kit. The downhole measurement assembly kit may include at least one of the first electrode 68, the second electrode 70, and/or the bracket 116 (e.g., described in FIG. 4) as discussed herein.

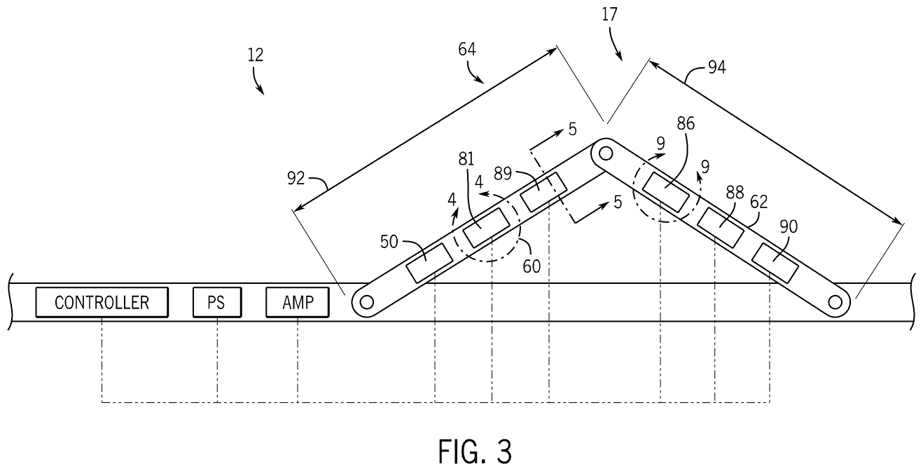

FIG. 3 is a side schematic view of the downhole measurement system 12 of FIG. 1 having multiple measurement assemblies 64 (e.g., measurement assemblies 80, 82, 84, 86, 88, and 90). In the illustrated embodiment, the measurement assemblies 80, 82, and 84 are coupled to the first arm 60 of the downhole caliper system 17 along a first length dimension 92 of the first arm 60. Additionally or alternatively, the measurement assemblies 86, 88, and 90 are coupled to the second arm 62 along a second length dimension 94 of the second arm 62. In certain embodiments, one or more measurement assemblies 64 may be coupled to the tool string 14. Although the illustrated embodiment shows a total of six measurement assemblies 64, the downhole measurement system 12 may include fewer or more measurement assemblies 64. For example, the downhole measurement system 12 may include 1, 2, 3, 4, 5, 7, 8, or measurement assemblies 64.

As shown, each of the measurement assemblies are separately wired to the controller 28. The measurement assemblies 64 may send separate measurements indicative of a capacitance of a fluid within the wellbore to the controller 28. In certain embodiments, the controller 28 may selectively power and/or receive measurements from a subset of the measurement assemblies 64. For example, the controller 28 may receive measurements from the measurement assemblies 86, 88, and 90, but may not receive measurements from the measurement assemblies 80, 82, and 84. As shown, the measurement assemblies 64 are electrically coupled to the same power source 38 and the same amplifier 66. In certain embodiments, the measurement assemblies 64 may be electrically coupled to multiple power sources and/or multiple amplifiers (e.g., via an electrical switch or multiplex components). In certain embodiments, one or more measurements may be taken at least partially sequentially or synchronously.

FIG. 4 is a perspective close-up view of one example of the measurement assembly 64 of the downhole measurement system 12 of FIG. 3 within an area identified by line 4-4. In the illustrated embodiment, the measurement assembly 64 may be described with respect to a longitudinal direction or axis 110, a radial direction or axis 112, and a circumferential direction or axis 114. As shown, the measurement assembly 64 includes the first electrode 68, the second electrode 70, and a bracket 116. The first electrode 68 is coupled to an inner surface 118 of an outer wall 120 (e.g., outer housing) of an arm 58 (e.g., arm portion) of the tool string.

As shown, the first electrode 68 includes a conductive plate 124 embedded into an insulative plate 126 composed of an insulator material (e.g., plastic, polyether ether ketone (PEEK), Teflon, oil-continuous fluid). As shown, the insulative plate 126 is coupled to the inner surface 118 of the outer wall 120 via fasteners 122. The conductive plate 124 includes a conductive surface 128 (e.g., front surface) that faces the second electrode 70. In the illustrated embodiment, the conductive plate 124 and the insulative plate 126 are both rectangular in shape. In certain embodiments, the conductive plate 124 and/or the insulative plate may be rounded, elliptical, square, or circular. In the illustrated embodiment, a first width dimension 125 of the insulative plate 126 is approximately 3 cm and a first length dimension 127 of the insulative plate 126 is approximately 4 cm. Additionally or alternatively, a second width dimension 129 of the conductive plate 124 is at least 2 cm, and a second length dimension 131 of the conductive plate 124 is at least 3 cm. In certain embodiments, the first width dimension 125 is at least 5 mm greater than the second width dimension 129. Additionally or alternatively, the first length dimension 127 may be at least 5 mm greater than the second length dimension 131.

As shown, the bracket 116 is coupled to the inner surface 118 via fasteners 122. The bracket 116 includes a first curved portion 130, a second curved portion 132, and a central portion 134 (e.g., portion, overlapping portion) disposed between and coupled to the first curved portion 130 and the second curved portion 132. The first curved portion 130 is coupled to the inner surface 118 at a first position 136 and the second curved portion 132 is coupled to the inner surface 118 at a second position 138. The first position 136 is offset and outward from a first end portion 140 of the first electrode 68 and the second position 138 is offset and outward from a second end portion 142 of the first electrode 68. As shown, the first position 136 and the second position 138 coincide with a first longitudinal central axis 144 of the first electrode 68 and the conductive surface 128. In certain embodiments, the first position 136 and/or the second position 138 may be offset from the first longitudinal central axis 144.

As shown, the second electrode 70 is coupled to a bracket inner surface 146 of the central portion 134 of the bracket 116. The bracket inner surface 146 faces the conductive surface 128 of the first electrode 68. As shown, the central portion 134 of the bracket 116 and the second electrode 70 are both disposed offset from the conductive surface 128 of the first electrode 68. In certain embodiments, the central portion 134 and the second electrode 70 are substantially parallel to the conductive surface 128. Additionally or alternatively, the central portion 134 and the second electrode 70 are at least partially disposed within an outer perimeter 148 of the conductive surface 128. For example, a plane 149 that intersects a second longitudinal central axis 150 of the second electrode 70 and the central portion 134 and is also normal (e.g., orthogonal) to the conductive plate 124 (e.g., and the insulative plate 126) intersects the conductive surface 128.

In the illustrated embodiment, the second longitudinal central axis 150 of the second electrode 70 is parallel to the first longitudinal central axis 144 of the first electrode 68 such that the plane 149 intersects both the first longitudinal central axis 144 and the second longitudinal central axis 150, while also being orthogonal (e.g., perpendicular) to the conductive surface 128. In certain embodiments, the second longitudinal central axis 150 may be skew and/or offset relative to the first longitudinal central axis 144. In certain embodiments, the first longitudinal central axis 144 and the second longitudinal central axis 150 may not be parallel with each other. Although the illustrated embodiment shows the second longitudinal central axis 150 of the second electrode 70 as being substantially parallel to the conductive surface 128, in certain embodiments the second longitudinal central axis 150 may not be parallel with the conductive surface 128.

In the illustrated embodiment, a direction of gravity 152 is substantially parallel with the conductive surface 128 and substantially perpendicular to the first longitudinal axis 144 and the second longitudinal axis 150. It may be appreciated that because the direction of gravity 152 does not point directly at the first electrode 68 nor the second electrode 70, a fluid 154 disposed in the wellbore 16 will be directed, via gravity, through a gap 156 between the pair of electrodes 67 such that buildup (e.g., coagulation) of sediment or suspended solids within the fluid 154 on the first electrode 68 and/or the second electrode 70 is mitigated.

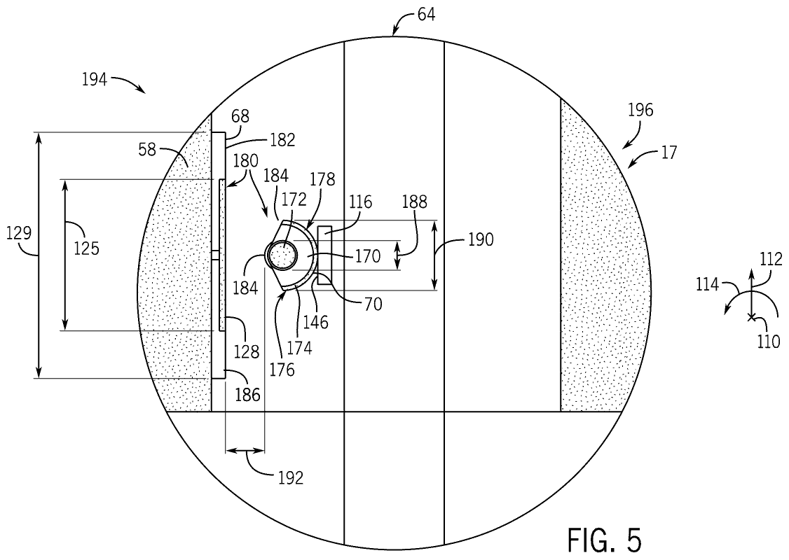

FIG. 5 is a cross-sectional view of the downhole measurement assembly 64 of FIG. 3 within an area identified by line 5-5 when the downhole caliper system 17 is in an open configuration. That is, the arms 58 of the downhole caliper system 17 are extended from the tool string. In the illustrated embodiment, the second electrode 70 includes an insulative housing 170 composed of an insulator material (e.g., plastic, PEEK, Teflon, oil-continuous fluid) and a conductive rod 172 (e.g., rod) composed of a conductive material (e.g., metal) disposed inside the insulative housing 170. Additionally or alternatively, the second electrode 70 includes a shielding layer 174 (e.g., electrical shielding layer) disposed in an outer radial portion 176 of the second electrode 70 partially about the conductive rod 172. As shown, the shielding layer 174 is disposed in a half sector 178 of the outer radial portion 176 disposed further away from the conductive surface 128 of the first electrode 68. As shown, the shielding layer 174 is disposed between the conductive rod 172 and the bracket inner surface 146 of the bracket 116.

In the illustrated embodiment, the first electrode 68 and the second electrode 70 are at least partially covered with insulator films 180 (e.g., insulator films 182 and 184). As shown, the insulator film 182 is disposed on the conductive surface 128 and an insulative surface 186 of the first electrode. Additionally, the insulator film 184 is disposed about the insulative housing 170 of the second electrode 70. In certain embodiments, either the insulator film 182 or the insulator film 184 may be omitted.

In the illustrated embodiment, a first diameter 188 of the conductive rod 172 of the second electrode 70 is between 1 millimeter (mm) and 10 mm. In certain embodiments, the first diameter 188 may be between 2 mm and 9 mm, 3 mm and 8 mm, or 4 mm and 7 mm. Additionally or alternatively, a second diameter 190 of the second electrode 70 is between 2 mm and 10 mm. In certain embodiments, the second diameter 190 is between 3 mm and 8 mm, 4 mm and 7 mm, or 5 mm and 6 mm. In certain embodiments, a first ratio between the first diameter 188 and the first width dimension 125 is between 1:6 and 4:5, 1:4 and 3:5, or 1:3 and 1:2. Additionally or alternatively, a second ratio between the first diameter 188 and the second width dimension 129 is between 1:6 and 4:5, 1:4 and 3:5, or 1:3 and 1:2.

In the illustrated embodiment, a distance 192 between the first electrode 68 and the second electrode 70 is greater than 3 mm. In certain embodiments, the distance 192 is between 1 mm and 8 mm, 2 mm and 6 mm, or 3 mm and 4 mm. In certain embodiments, a third ratio between the distance 192 and the second diameter 190 of the second electrode is between 1:7 and 4:5. In the illustrated embodiment, the downhole measurement assembly 64 is disposed on a left side 194 of the arm 58. In certain embodiments, the downhole measurement assembly 64 may be disposed on the left side 194 of the arm 58, a right side 196 of the arm 58, or a combination thereof. Additionally or alternatively, the downhole measurement assembly 64 may be disposed at another location in the tool string (e.g., another arm).

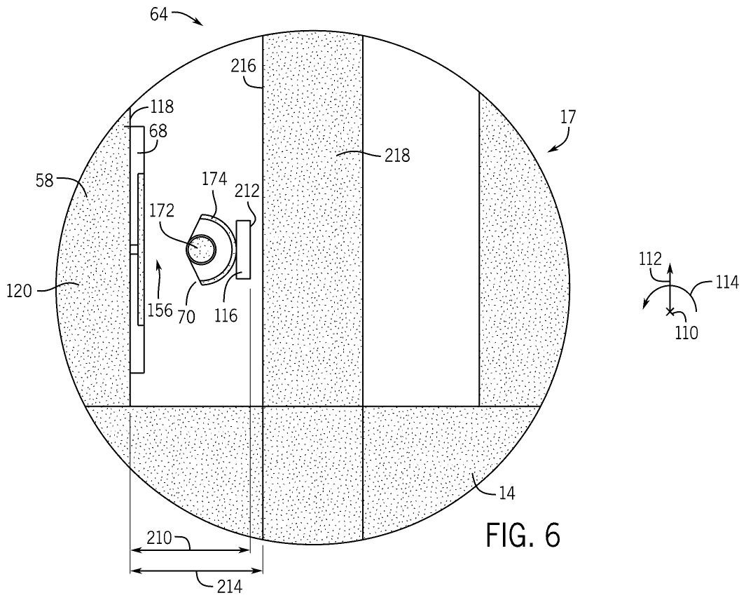

FIG. 6 is a cross-sectional view of the downhole measurement assembly 64 of FIG. 3 with the downhole caliper system 17 in a closed configuration. When the downhole caliper system 17 is in the closed configuration, the arms 58 of the downhole caliper system 17 are retracted into the tool string 14. In the illustrated embodiment, a distance 210 between the inner surface 118 of the outer wall 120 of the arm 58 and a bracket outer surface 212 of the bracket 116 is less than a clearance distance 214 between the inner surface 118 and an outer surface 216 of a middle section 218 (e.g., spine) of the tool string 14, such that the measurement assembly 64 clears the middle section 218, thereby enabling the arm 58 to retract into the tool string 14.

It may be appreciated that the shielding layer 174 of the second electrode 70 disposed about the conductive rod 172 may mitigate fluctuation of the signal received by the controller due to proximity of the middle section 218 of the tool string 14 during the closed configuration. That is, the presence of the shielding layer 174 at the back of the second electrode 70 may reduce the sensitive region of the pair of electrodes 67 to the gap 156 (e.g., region) disposed between the first electrode 68 and the second electrode 70.

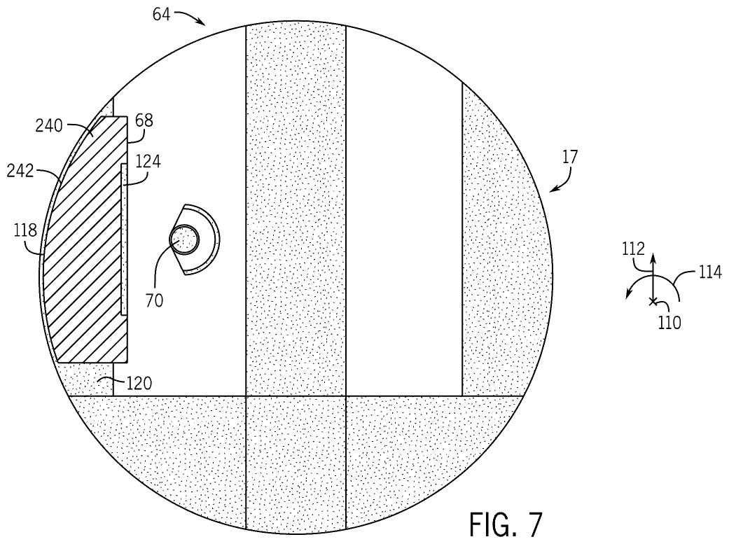

FIG. 7 is a cross-sectional view of the downhole measurement assembly 64 of FIG. 3 with the downhole caliper system 17 in the closed configuration and the first electrode 68 of the downhole measurement assembly 64 formed into the tool string 14. In the illustrated embodiment, the first electrode 68 includes an insulative insert 240 and the conductive plate 124. As shown, the conductive plate 124 is embedded into the insulative insert 240. In the illustrated embodiment, the insulative insert 240 includes a curved outer side 242 that is coupled to the outer wall 120 of the arm 58. That is, the curved outer side 242 may include a curved shape that substantially matches a shape of the inner surface 118 of the outer wall 120. In certain embodiments, the insulative insert 240 may integrally form a portion of the outer wall 120. That is, a portion of the outer wall 120 may be formed of insulative material into which the conductive plate 124 is embedded.

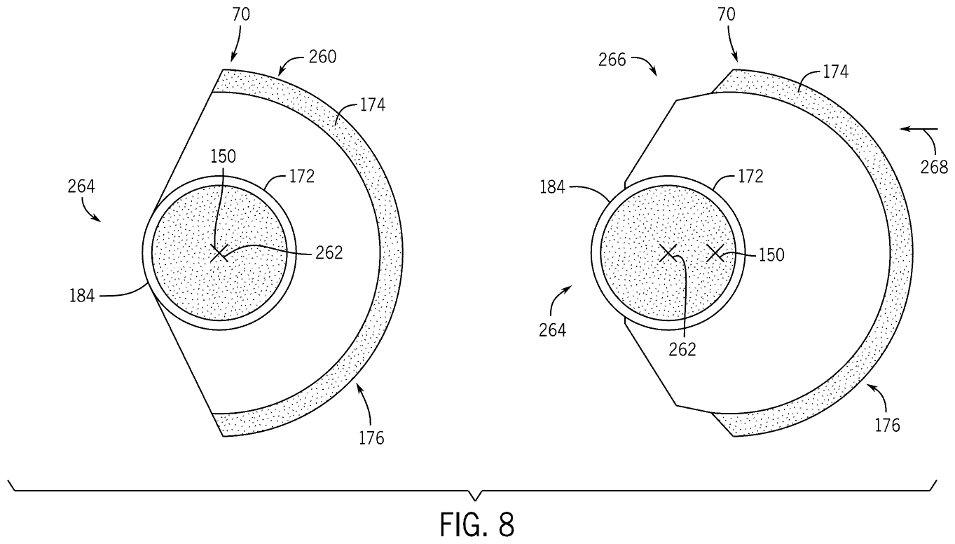

FIG. 8 is a series of cross-sectional views of the second electrode 70 of the downhole measurement assembly. In the view 260, a third longitudinal central axis 262 of the conductive rod 172 is colinear (e.g., aligns with) the second longitudinal central axis 150 of the second electrode 70. As shown, the second electrode 70 includes the shielding layer 174 disposed about the outer radial portion 176 of the second electrode 70. In the illustrated embodiment, the second electrode 70 includes the insulator film 184 disposed on a sensing portion 264 of the second electrode 70.

In the view 266, the third longitudinal central axis 262 of the conductive rod 172 is offset from the second longitudinal central axis 150 of the second electrode 70. In the illustrated embodiment, the third longitudinal central axis 262 is offset from the second longitudinal central axis 150 in a radial direction 268, toward the first electrode. In certain embodiments, the third longitudinal central axis 262 may be offset from the second longitudinal central axis 150 in another direction. As shown, the second electrode 70 includes the shielding layer 174 disposed about the outer radial portion 176 of the second electrode 70. In the illustrated embodiment, the second electrode 70 includes the insulator film 184 disposed on a sensing portion 264 of the second electrode 70.

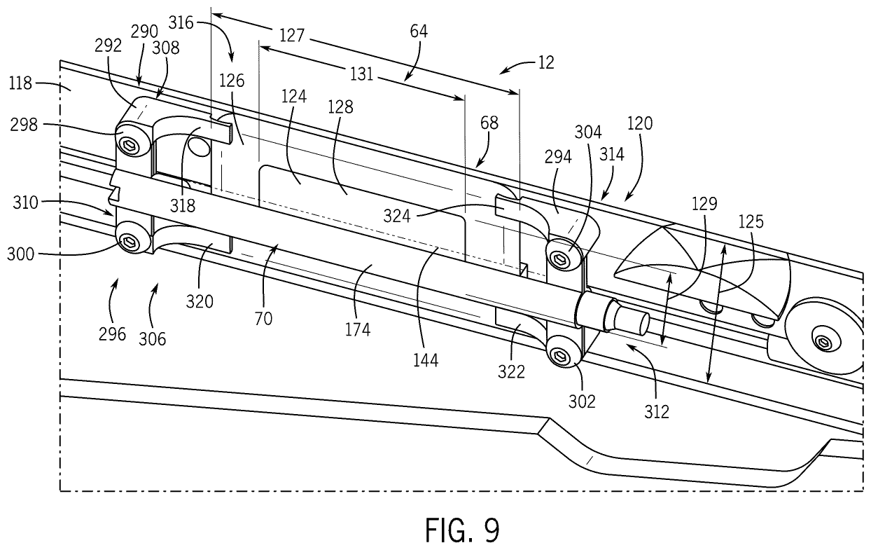

FIG. 9 is a perspective close-up view of the downhole measurement assembly 64 of the downhole measurement system 12 of FIG. 3 within an area identified by line 9-9. In the illustrated embodiment, the first electrode 68 includes the conductive plate 124 embedded into the insulative plate 126 composed of an insulator material (e.g., plastic, polyether ether ketone (PEEK), Teflon, oil-continuous fluid). As shown, the insulative plate 126 is coupled to the inner surface 118 of the outer wall 120. The conductive plate 124 includes a conductive surface 128 (e.g., front surface) that faces the second electrode 70. In the illustrated embodiment, the conductive plate 124 and the insulative plate 126 are both rectangular in shape. In certain embodiments, the conductive plate 124 and/or the insulative plate may be rounded, elliptical, square, or circular. In the illustrated embodiment, the first width dimension 125 of the insulative plate 126 is approximately 3 cm and the first length dimension 127 of the insulative plate 126 is approximately 4 cm. Additionally or alternatively, the second width dimension 129 of the conductive plate 124 is at least 2 cm, and the second length dimension 131 of the conductive plate 124 is at least 3 cm. In certain embodiments, the first width dimension 125 is at least 5 mm greater than the second width dimension 129. Additionally or alternatively, the first length dimension 127 may be at least 5 mm greater than the second length dimension 131.

In the illustrated embodiment, the second electrode 70 is retained via brackets 290 (e.g., first bracket 292, second bracket 294). As shown, the brackets 290 are coupled to the inner surface 118 of the outer wall 120 via fasteners 296 (e.g., fasteners 298, 300, 302, and 304) at locations 306 (e.g., locations 308, 310, 312, and 314). As shown, the locations 306 are disposed longitudinally outward (e.g., offset) from the first electrode 68, as well as offset from the first longitudinal central axis 144 of the first electrode 68.

In the illustrated embodiment, the brackets 290 include overhang portions 316 (e.g., overhang portions 318, 320, 322, and 324) that at least partially couple the insulative plate 126 to the inner surface 118 of the outer wall 120. As shown, the second electrode 70 is coupled to the first bracket 292 and the second bracket 294. In the illustrated embodiment, the shielding layer 174 of the second electrode 70 mechanically supports the second electrode 70 between the first bracket 292 and the second bracket 294.

Technical effects include estimating a capacitance of a fluid disposed in a wellbore in order to estimate a composition of the fluid (e.g., water-continuous, oil continuous). The orientation of the measurement assembly mounted to the arm of the tool string relative to the direction of gravity, as well as the shape and size of the electrodes, mitigate the risk of clogging of the fluid between the electrodes. The covering of the electrodes with an insulator film (e.g., Teflon) mitigates fouling of the electrodes caused by deposited material from the fluid. Finally, the electrical shielding disposed about the back side of the second electrode between the conductive rod and the tool string spine mitigates fluctuation of the measurement caused by the tool string spine, thereby ensuring the sensor measurement is independent of the arm opening angle.

The subject matter described in detail above may be defined by one or more clauses, as set forth below.

According to a first aspect, a system includes an arm portion of a tool string to be lowered into a wellbore and a measurement system. The measurement system includes a first electrode coupled to an inner surface of the arm portion, a first bracket coupled to the inner surface, and a second electrode coupled to a portion of the first bracket. The second electrode and the portion of the bracket are offset from a conductive surface of the first electrode. The second electrode and the portion of the first bracket are at least partially within an outer perimeter of the conductive surface.

The system of the preceding clause, wherein the second electrode is a transmitter electrode configured to output a signal and the first electrode is a receiver electrode configured to detect the signal.

The system of any preceding clause, wherein the first electrode is a transmitter electrode configured to output a signal and the second electrode is a receiver electrode configured to detect the signal.

The system of any preceding clause, wherein the first electrode includes a plate, the plate includes the conductive surface, and the second electrode comprises: a housing comprising an insulation material; and a rod having a conductive material, wherein the rod is disposed inside the housing.

The system of any preceding clause, wherein a ratio between a diameter of the second electrode and a width of the first electrode is between 1:4 and 3:5.

The system of any preceding clause, wherein a ratio between a distance spanning from the first electrode to the second electrode and the diameter of the second electrode is between 1:7 and 4:5.

The system of any preceding clause, wherein the second electrode is coupled to an inner surface of the first bracket, wherein the inner surface faces the conductive surface.

The system of any preceding clause, wherein the second electrode includes an electrical shielding layer at least partially disposed about the rod, wherein the electrical shielding layer is disposed between the rod and the inner surface of the first bracket.

The system of any preceding clause, wherein the first bracket includes first and second curved portions; and the portion, wherein the portion is coupled to the first and second curved portions and disposed between the first and second curved portions.

The system of any preceding clause, including a second bracket, wherein the first bracket is coupled to the inner surface at a first position, the second bracket is coupled to the inner surface at a second position, the first and second positions are offset from the first electrode, and the second electrode is coupled to the first and second brackets.

According to a second aspect, a downhole measurement system includes a first electrode that couples to an inner surface of an arm portion of a tool string, a bracket that couples to the inner surface, and a second electrode that couples to a portion of the bracket. The second electrode and the portion of the bracket, when coupled to the inner surface, are offset from a conductive surface of the first electrode. The second electrode and the portion, when coupled to the inner surface, are disposed within an outer perimeter of the conductive surface.

The system of the preceding clause, wherein the second electrode is a transmitter electrode configured to output a signal and the first electrode is a receiver electrode configured to detect the signal.

The system of any preceding clause, wherein an operating frequency of the first electrode, the second electrode, or a combination thereof is between 20 kilohertz and 10 megahertz.

The system of any preceding clause, wherein an insulator film is disposed on a first front face of the first electrode, a housing of the second electrode, or a combination thereof.

The system of any preceding clause, wherein the first electrode is electrically coupled to an electrical source via a first wire and the second electrode is separately electrically coupled to the electrical source via a second wire.

The system of any preceding clause, wherein a first length of the first wire, a second length of the second wire, or a combination thereof is greater than 50 centimeters, and the first wire is a shielded wire that couples to an amplifier.

The system of any preceding clause, including a controller having a memory and a processor, wherein the controller receives a signal from the first electrode indicative of a capacitance, a dielectric permittivity, a conductivity, or a combination thereof of a fluid disposed between the first and second electrodes; and determines an estimated capacitance of the fluid based on the received signal.

According to a third aspect, a system includes a downhole measurement assembly kit. The downhole measurement assembly kit includes at least one of a first electrode that couples to an inner surface of an arm portion of a tool string, a bracket that couples to the inner surface, and a second electrode that couples to a portion of the bracket. The second electrode and the portion of the bracket, when coupled to the inner surface, are offset from a conductive surface of the first electrode. The second electrode and the portion of the bracket, when coupled to the inner surface, are within an outer perimeter of the conductive surface.

The system of the preceding clause, wherein the first electrode is a receiver electrode and the second electrode is a transmitter electrode.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. Moreover, the order in which the elements of the methods described herein are illustrated and described may be re-arranged, and/or two or more elements may occur simultaneously. The embodiments were chosen and described in order to best explain the principals of the disclosure and its practical applications, to thereby enable others skilled in the art to best utilize the disclosure and various embodiments with various modifications as are suited to the particular use contemplated.

Finally, the techniques presented and claimed herein are referenced and applied to material objects and concrete examples of a practical nature that demonstrably improve the present technical field and, as such, are not abstract, intangible or purely theoretical.

1. A system, comprising:
an arm portion of a tool string configured to be lowered into a wellbore; and
a measurement system, comprising:
a first electrode coupled to an inner surface of the arm portion;
a first bracket coupled to the inner surface; and
a second electrode coupled to a portion of the first bracket, wherein the second electrode and the portion of the first bracket are:
offset from a conductive surface of the first electrode; and
at least partially disposed within an outer perimeter of the conductive surface.

2. The system of claim 1, wherein the second electrode is a transmitter electrode configured to output a signal and the first electrode is a receiver electrode configured to detect the signal.

3. The system of claim 1, wherein the first electrode is a transmitter electrode configured to output a signal and the second electrode is a receiver electrode configured to detect the signal.

4. The system of claim 2, wherein the first electrode comprises a plate, the plate comprises the conductive surface, and the second electrode comprises:
a housing comprising an insulation material; and
a rod comprising a conductive material, wherein the rod is disposed inside the housing.

5. The system of claim 4, wherein a first longitudinal central axis of the first electrode is parallel with a second longitudinal central axis of the second electrode, and a plane intersecting both the first and second longitudinal central axes is orthogonal to the conductive surface.

6. The system of claim 4, wherein a ratio between a diameter of the second electrode and a width of the first electrode is between 1:4 and 3:5.

7. The system of claim 6, wherein a ratio between a distance spanning from the first electrode to the second electrode and the diameter of the second electrode is between 1:7 and 4:5.

8. The system of claim 4, wherein the second electrode is coupled to an inner surface of the first bracket, wherein the inner surface faces the conductive surface.

9. The system of claim 8, wherein the second electrode comprises an electrical shielding layer at least partially disposed about the rod, wherein the electrical shielding layer is disposed between the rod and the inner surface of the first bracket.

10. The system of claim 5, wherein the first bracket comprises:
first and second curved portions; and
the portion, wherein the portion is coupled to the first and second curved portions and disposed between the first and second curved portions.

11. The system of claim 1, comprising a second bracket, wherein the first bracket is coupled to the inner surface at a first position, the second bracket is coupled to the inner surface at a second position, the first and second positions are offset from the first electrode, and the second electrode is coupled to the first and second brackets.

12. A downhole measurement system, comprising:
a first electrode configured to couple to an inner surface of an arm portion of a tool string;
a bracket configured to couple to the inner surface; and
a second electrode configured to couple to a portion of the bracket, wherein the second electrode and the portion of the bracket, when coupled to the inner surface, are:
offset from a conductive surface of the first electrode; and
at least partially disposed within an outer perimeter of the conductive surface.

13. The system of claim 12, wherein the second electrode is a transmitter electrode configured to output a signal and the first electrode is a receiver electrode configured to detect the signal.

14. The system of claim 13, wherein an operating frequency of the first electrode, the second electrode, or a combination thereof is between 20 kilohertz and 10 megahertz.

15. The system of claim 13, wherein an insulator film is disposed on a first front face of the first electrode, a housing of the second electrode, or a combination thereof.

16. The system of claim 13, wherein the first electrode is electrically coupled to receiver circuitry via a first wire and the second electrode is separately electrically coupled to transmitter circuitry via a second wire.

17. The system of claim 16, wherein a first length of the first wire, a second length of the second wire, or a combination thereof is greater than 50 centimeters, and the first wire is a shielded wire configured to couple to an amplifier.

18. The system of claim 16, comprising a controller having a memory and a processor, wherein the controller is configured to:
receive a signal from the first electrode indicative of a capacitance, a dielectric permittivity, a conductivity, or a combination thereof of a fluid disposed between the first and second electrodes; and
determine an estimated capacitance of the fluid based on the received signal.

19. A system, comprising:
a downhole measurement assembly kit, comprising at least one of:
a first electrode configured to couple to an inner surface of an arm portion of a tool string;
a bracket configured to couple to the inner surface; and
a second electrode configured to couple to a portion of the bracket, wherein the second electrode and the portion of the bracket, when coupled to the inner surface, are:
offset from a conductive surface of the first electrode; and
at least partially disposed within an outer perimeter of the conductive surface.

20. The system of claim 19, wherein the first electrode is a receiver electrode and the second electrode is a transmitter electrode.
